(19) United States
(12) Patent Application Publication
(10) Pub. No.: US 2006/0057216 A1
(43) Pub. Date: Mar. 16, 2006

(54) Low-obscuration image transmitting particulate ocular therapeutic formulations

(76) Inventors: Joseph C. Salamone, Fairport, NY (US); Adrian T. Raiche, Fairport, NY (US)

Correspondence Address: Bausch & Lomb Incorporated, One Bausch & Lomb Place, Rochester, NY, 14604-2701, US

(21) Appl. No.: 10/941,389

(22) Filed: Sep. 15, 2004

(51) Int. Cl. A61K 9/14

(52) U.S. Cl. 424/489; 424/427

(57) ABSTRACT

Low-obscuration image transmitting particulate ocular therapeutic formulations are described. Additionally, methods are described for producing and for using such low-obscuration image transmitting particulate ocular therapeutic formulations.

### FIELD OF THE INVENTION

The present invention relates to the reduction of therapeutic particle or particulate induced light reflection, refraction and diffraction in eye by using material formulations produced optimizing size/refractive index/concentration interactions. More particularly, the present invention relates to ocular therapeutic formulations with optimized formulation particulate light reflection, refraction and diffraction interactions thereby improving patient vision during ocular therapeutic treatment.

### BACKGROUND OF THE INVENTION

Eye medication is typically administered for one of two purposes—to treat the exterior of the eyes for infections such as conjunctivitis, blepharitis and keratitis sicca, or to treat the interior of eyes, i.e., intraocular treatment, for diseases such as glaucoma or uveitis. Most ocular diseases are treated through topical applications of solutions administered as eye drops. One major problem encountered with topical delivery of ophthalmic drugs or therapeutic agents is the rapid and extensive loss of drug through drainage and high tear fluid turn over. After instillation of an eye-drop in an eye, typically less than 2 to 3 percent of the applied drug penetrates the cornea. A major fraction of such instilled doses are often absorbed systematically via the conjunctiva and nasolacrimal duct. Another limitation encountered with topical delivery is a relatively impermeable corneal barrier that limits ocular absorption.

Ocular drug delivery attempts to control and ultimately optimize the delivery of therapeutic agents or drugs to their target tissues within the eye. In the past, ocular drug delivery commonly was accomplished by encapsulating therapeutic agents in both degradable and non-degradable polymers in forms of plaques, rods, tubes and spherical and non-spherical particles of varying dimensions. Such particles or objects strongly scatter electromagnetic radiation having wavelengths less than or equal to nominal object size. The accepted range of the band of the electromagnetic spectrum known as visible light is 400 to 700 nm. Objects lying in or moving into the path of light reflected from a second object and the focusing area of the retina will in part or in whole obstruct the view of the second object. Extent of obscuration is proportional to absorption refractive index coefficient of an object, deviation of real object refractive index from suspending medium, deviation in object size compared to incident wavelengths, and number of scattering events. Such vision obstruction is undesirable in ocular drug delivery.

### SUMMARY OF THE INVENTION

The present invention relates to optimized formulations for minimized therapeutic particulate induced light reflection, refraction and diffraction upon ophthalmic use. Formulations of the present invention minimize therapeutic particulate induced light reflection, refraction and diffraction by manipulating and controlling size/refractive index coupled interactions to thus reduce vision obstruction while affecting mass delivery of therapeutic agent and improve patient vision during ocular therapeutic treatment.

The subject therapeutic formulations are effective in the delivery of therapeutically effective amounts of one or more therapeutically active agents while minimizing patient vision obstruction during use. The subject formulations are likewise biocompatible, causing little or no tissue irritation.

Accordingly, it is an object of the present invention to provide ophthalmic therapeutic formulations that minimize or eliminate vision obstruction during use.

Another object of the present invention is to provide a method for the production of therapeutic formulations useful in ophthalmic applications.

Another object of the present invention is to provide a method for the production of therapeutic formulations containing a therapeutically effective amount of a therapeutically active agent.

Another object of the present invention is to provide a method for the production of biocompatible formulations for ophthalmic drug delivery.

Another object of the present invention is to provide a method for the production of biocompatible formulations for ophthalmic drug delivery without or with minimal eye irritation.

Still another object of the present invention is to provide a method for the production of therapeutic formulations useful in ophthalmic applications without or with minimal visual acuity alteration.

These and other objectives and advantages of the present invention, some of which are specifically described and others that are not, will become apparent from the detailed description and claims that follow.

### BRIEF DESCRIPTION OF THE DRAWINGS

FIG. 1 is a polar plot of logarithmic values of intensity for external reflection and diffraction, direct transmission and internal reflection scattering phenomena;

FIG. 2 is a polar plot of logarithmic values of intensity for different values of refractive index;

FIG. 3 is a polar plot of logarithmic values of intensity for different values of imaginary portion of refractive index;

FIG. 4 is a polar plot of logarithmic values of intensity for different diameter particles of 1.0 refractive index and light of 555 nm;

FIG. 5 is a graph illustrating Steven's Law for the limit of particle concentrations as a function of real refractive index for varying particle diameters;

FIG. 6 is a graph illustrating maximum allowable particle concentration as a function of diameter for a given refractive index;

FIG. 7 is a graph illustrating maximum allowable particle concentration as a function of imaginary refractive index for 1 μm particles having real refractive indices of 1.3 and 1.8; and

FIG. 8 us a graph illustrating maximum allowable particle concentration as a function of real refractive index for varying levels of incident intensity for 1 μm diameter particles.

### DETAILED DESCRIPTION OF THE INVENTION

Light interacts with materials of all sizes creating reflection, refraction, and diffraction. Light scattering resulting from reflective, refractive and diffractive interactions produces spherical fluctuations in intensity. Particulate compounding of these fluctuations or variations in intensities results in an inability to focus a complete image on the retina, i.e., vision obstruction. In accordance with the present invention, by varying ophthalmic formulation particulate size, particulate components, and the major contributors to reflection, refraction, and diffraction, i.e., particulate diameter, real refractive index, and imaginary refractive index, the number of particulates allowable in the path of light, without causing noticeable attenuation in the light used to form an image, may be increased.

The eye's cornea and lens act in coordinated effort to force convergence of light. Light striking the surface of the cornea may have a divergent angle of at least approximately 0.44 degrees up to collimated light. Light passing between the cornea and lens and between the lens and retina is convergent. A particulate obstructing the path of light prior to reaching the cornea will reduce intensity of light over an area no less than its cross-sectional area. This area of reduced light intensity upon reaching the retina is at least equivalent in relative cross-sectional area to objects in a plane having a vector orthogonal to the corneal surface. A particulate obstructing the path of light between the lens and retina will reduce the intensity of light over an area less than its cross-sectional area.

Only approximately one percent of light striking the cornea, contacts the retina. The eye's iris helps regulate light intensity entering the eye by varying pupil diameter from about 1 mm to about 5 mm. In addition to this mechanical light regulation by the iris, photoreceptor cells are modulated by their resting polarization level to adapt the eye to maximize sensitivity. Photoreceptor cells vary in wavelength sensitivity and distribution across the retina. Sensation from individual photoreceptor cells is integrated both by time and space into a single signal to the brain. Photoreceptor cells are most dense in the fovea centralis. The fovea centralis converges into a zonule covering an area of approximately 0.06 degrees on the retina. This translates to a zonule body of approximately 13 μm in diameter at the most sensitive part of the eye. Outside of the fovea centralis, sensitivity drops precipitously. Actions of photoreceptors within a zonule coordinate image acquisition, contrast, and motion. The first occurs predominantly in the fovea where color photoreceptors are present, and the latter occur in the surrounding areas. Intensity of light entering the eye is, to a point, directly proportional to visual acuity. Under low light conditions, less than 0.0318 candelas/m², where contrast and motion are used to identify visual targets, best attainable visual acuity is approximately 20/80. Under brightening conditions, visual acuity improves from 20/80 to approximately 20/12 at 300 candelas/m².

Change in light intensity detectable by the eye is described by Steven's law. Steven's law outlines a power law response of change in visual perception as a function of intensity of visual perception. Because eyes adapt to changing light conditions, a fixed system of a required number of photons to create sensation of increased or decreased brightness would lead to a lack of sensitivity under dim light and hypersensitivity under bright light. Over the range of well-lit conditions, Steven's law can be simplified to: a 1 dB or 10% change in intensity is the just noticeable difference in detectability.

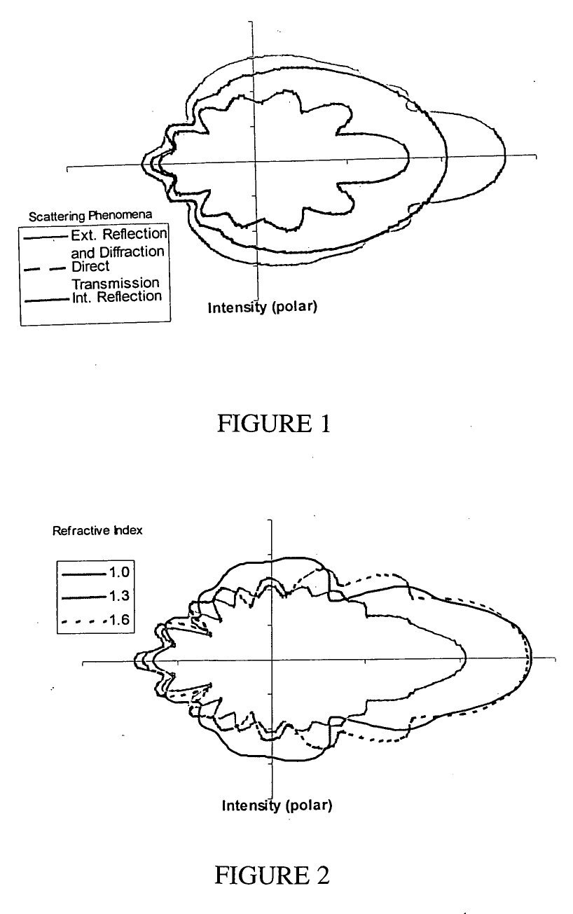

Light impinging on particles is reflected, refracted, diffracted or absorbed to different extents based on varying particulate size, real refractive index difference, and imaginary refractive index difference. Rigorous solutions for resulting patterns of light intensity are provided by, among others, Debye infinite series. Debye infinite series differ from other methods of calculating light scattering by separating external reflection and diffraction from transmitted light from internal reflection. All of these contribute to the total light scattering result as is illustrated in FIG. 1.

The difference of particle refractive index compared to surrounding medium significantly alters light scattering. Total light scattering from a 1 μm particulate with a real refractive index of 1.3 in vitreous humor (real refractive index of 1.337) has the smallest light scattering profile. As the refractive index is increased to 1.6, a value greater than the vitreous, it is largely forward scattering that increases. When particle refractive index is decreased to 1.0, forward scattering increases and more light is projected to the sides of the particulate as the likelihood of total internal reflectance increases as is illustrated in FIG. 2.

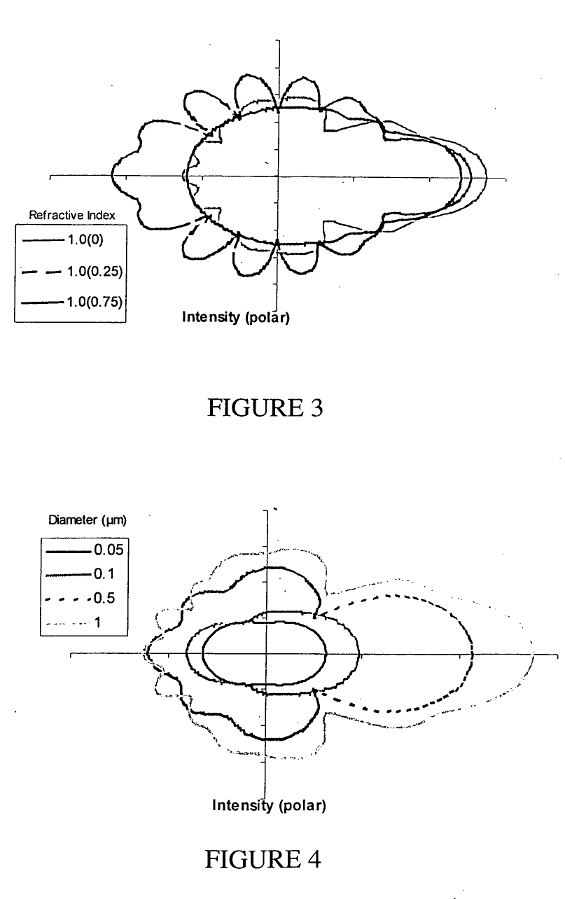

Refractive indexes are made up of two values, the real part is the measure the speed of light passage through the material, and the imaginary part is the absorbance of light by the material. In addition to the effect of light scattering by the real part previously demonstrated, absorbance of light also plays a critical role in light scattering. A small increase in the imaginary refractive index from 0 to 0.25, leaves forward scattering largely unaffected, but significantly increases the amount of light reflected off the incidence surface of the sphere as illustrated in FIG. 3. As the imaginary refractive index further increases, this increased backscattering decreases substantially.

Light scattering is also significantly dependent on particulate diameter. When a particulate is much smaller than the wavelength of light, scattering is minimal as illustrated in FIG. 4. As particulate diameter approaches and exceeds the wavelength of impinging light, scattering significantly increases in all directions.

Light reflected off an object entering the eye will be scattered by particulate residing in the path that light. For an entering beam of light of wavelength 555 nm, for which the eye is maximally sensitive in a well-lit environment, intensity variations due to light scattering were calculated to a resolution of 0.05° and an incident intensity of 300 lumens/m². The number of particulates required to reduce forward scattering of light intensity by 10% (1 dB), the just noticeable limit of luminance detection, were determined for different values of real and imaginary refractive indexes and particulate size diameters.

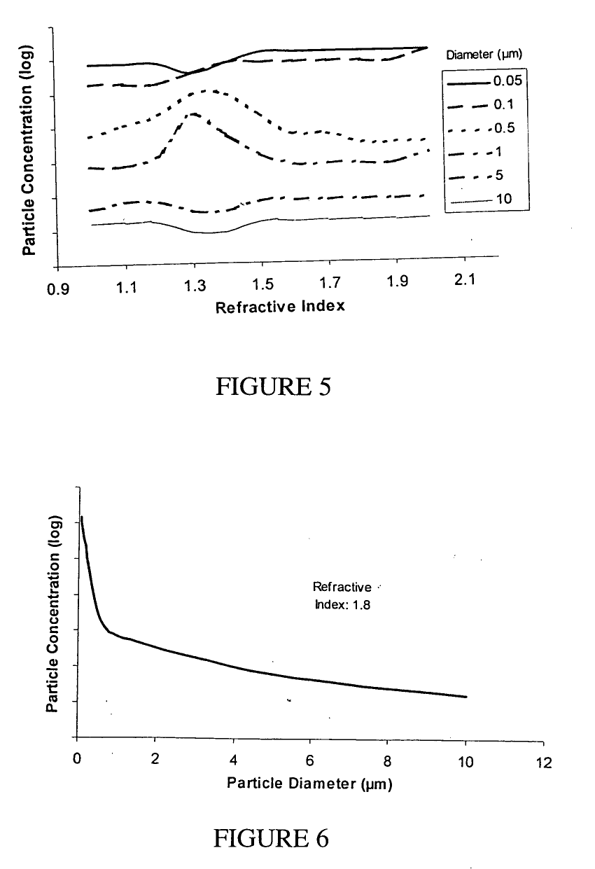

Maximum allowable particulate concentration fell into three categories depending on whether particulate diameters were substantially smaller than, on the order of, or substantially larger than the wavelength of incident light as illustrated in FIG. 5. For particulates with diameters substantially smaller than 555 nm, when particulate real refractive index was less than vitreous humor, the maximum allowable particulate concentration was slightly less than when the refractive index was greater than vitreous. This is due to increased occurrence of total internal reflection; the critical angle for total internal reflection decreases as real refractive index decreases relative to surrounding media. When particulate diameters were similar to wavelength of incident light, maximum allowable particulate concentration was greatest when particle real refractive index was near the refractive index of the vitreous and decreased nonlinearly away from the acme. As particulate diameter increased away from the wavelength, the rate of decrease in maximum allowable particulates decreased at an increasing rate. When particulate diameters were substantially greater than the wavelength of incident light, maximum allowable particulate concentration was decreased compared to previous cases.

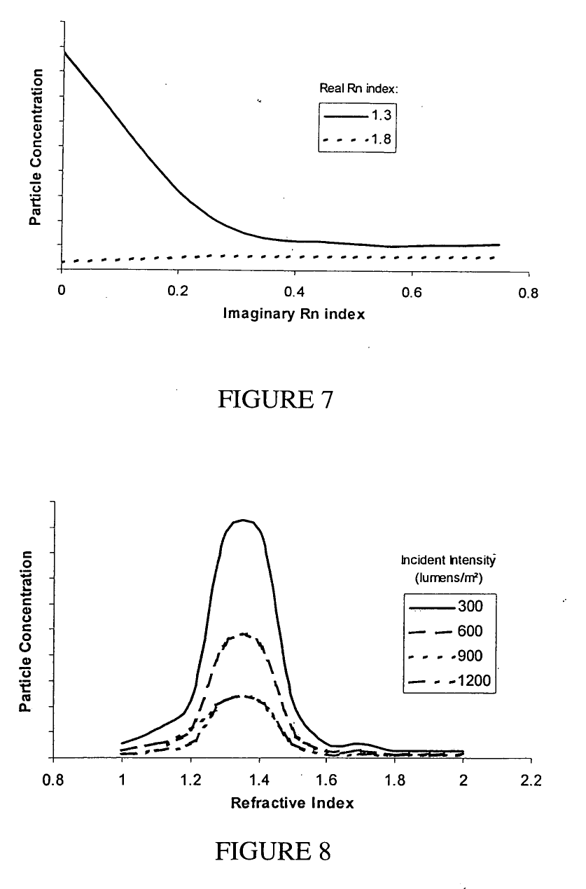

Maximum allowable particulate concentration as a function of particulate size for a given refractive index is non-linearly, inversely proportional to particulate diameter as illustrated in FIG. 6. For a given particulate diameter, changing the imaginary refractive index for a particulate having a real index of refraction close to the vitreous does not substantially affect the maximum allowable particulate concentration as illustrated in FIG. 7. When particulate real refractive index is substantially different from the vitreous, any contribution to absorbance by the imaginary refractive index reduces maximum allowable particulate concentration to a constant level.

Because scattering intensity is a function of incident intensity, maximum allowable particulate concentration decreases as intensity increases. However, as intensity continues to increase, a plateau is eventually reached where a range of angles exceeds the just noticeable difference. For 1 μm particles, there was not substantial difference in maximum allowable particulate concentration when incident light intensity was 900 and 1200 lumens/m² as illustrated in FIG. 8.

Formulations of the present invention maximize image transmittance of ocular therapeutic treatments by optimizing particulates forming the ocular therapeutic treatments. Ocular therapeutic formulation particulates of the present invention for use in topically applied ophthalmic therapeutic formulations preferably have an imaginary refractive index of less than about 0.20 or greater than about 0.30, more preferably less than about 0.15 or greater than about 0.50, and most preferably less than about 0.10 or greater than about 0.65. The particles likewise preferably have a real refractive index of about 1.0 to about 1.8, more preferably of about 1.1 to about 1.6, most preferably from about 1.2 to about 1.4. Preferable diameters for such particulates are from about 0.10 nm to less than about 500 nm, more preferably from about 0.25 nm to about 375 nm, and most preferably from about 0.50 nm to about 350 nm for use in therapeutic formulations for topically applied ocular treatments at a concentration of about 0.01 to about 500 mg/ml, more preferably of about 0.1 to about 100 mg/ml, and most preferably of about 1 to about 10 mg/ml.

Ocular therapeutic formulation particulates of the present invention for use in the anterior or posterior segments of an eye preferably have an imaginary refractive index of less than about 0.20 or greater than about 0.30, more preferably less than about 0.15 or greater than about 0.50, and most preferably less than about 0.10 or greater than about 0.65. The particles likewise preferably have a real refractive index of about 1.0 to about 1.8, more preferably of about 1.1 to about 1.6, most preferably from about 1.2 to about 1.4. Preferable diameters for such particulates are from about 0.10 nm to less than about 500 nm, more preferably from about 0.25 nm to about 375 nm, and most preferably from about 0.50 nm to about 350 nm for use in therapeutic formulations for the anterior or posterior segments of an eye at a concentration of about 0.01 to about 500 mg/ml, more preferably of about 0.1 to about 100 mg/ml, and most preferably of about 1 to about 10 mg/ml.

In optimizing the low-obscuration image transmitting particulate formulations of the present invention, one or more secondary materials may be added to the particulates as a coating or by alternative means to alter the diameter, real refractive index, imaginary refractive index, or a combination thereof, of the particulates. Suitable secondary materials include for example but are not limited to polyolefins, polyethers, polyesters, polyanhydrides, polyamides, polyorthoesters, polyacrylates, polysulfones, polysiloxanes, polysaccharides, polyvinylpyrrolidones, polyols, alcohols, water and minerals.

The ocular therapeutic formulation particulates of the present invention are useful for the delivery of drugs in a targeted fashion through topical application or through delivery directly into the eye, such as for example by injection, without alteration of vision. The desired drug may be so delivered to the anterior chamber of the eye or the posterior chamber of the eye including for example the vitreous, intraretinal space, subretinal space, intrachoroidal space and the suprachoroidal space. The subject drug delivery formulations are useful for the control or reversal of diseases of the posterior of the eye including for example but not limited to glaucoma, uveitis, age-related macular degeneration, retinitis pigmentosa, diabetic macular edema, nonproliferative and proliferative diabetic retinopathy, idiopathic premacular fibrosis, Terson syndrome, VX2 intraocular tumors and endophthalmitis.

Methods for producing low-obscuration image transmitting ocular therapeutic formulation particulates in accordance with the present invention can be accomplished through either one of two routes. One route is a process in which one or more therapeutically active agents are not soluble in one or more solvents or solvent system in which one or more therapeutically inactive agents are soluble. Examples of such a process include for example but are not limited to fluidized bed coating, precipitation of therapeutically inactive coatings from solution, polymerization of therapeutically inactive coatings and spraying or co-spraying of therapeutically active and inactive agents as described in more detail in the examples below. Another route is a process in which both one or more therapeutically active agents and one or more therapeutically inactive agents are soluble in one or more solvents or solvent system. Examples of such a process include for example but are not limited to nanoprecipitation, spray drying, emulsification, and all forms of solvent removal into liquid or gas phase or monomer polymerization as described in more detail in the examples below. includes several critical components.

Critical components to the preferred method of the present invention include: 1) a solvent miscible or soluble in a non-solvent; 2) a solvent/non-solvent system in which the polymer or matrix is soluble; 3) a ternary agent soluble in the non-solvent and the solvent/non-solvent system but not soluble in the solvent; 4) a solvent having temperature dependent solubility in a solution of the non-solvent and ternary agent; and 5) a surfactant soluble in the non-solvent and solvent/non-solvent system but not soluble in the solvent. Such polymeric particles made in accordance with the present invention with therapeutically effective amounts of therapeutically active agents incorporated therein are produced using an agent solvent that: 1) is miscible or soluble in a non-solvent; 2) is not a good solvent for a ternary agent that is soluble in the non-solvent; 3) has temperature dependent solubility in a solution of the non-solvent and ternary agent; 4) is not a solvent for a surfactant that is soluble in the non-solvent; and 5) is part of a solvent/non-solvent system that is a solvent for one or more therapeutically active agents to be incorporated. The solvent for the therapeutically active agent(s) or “agent solvent” may be identical to or different than the solvent for the polymer or matrix. The solvent for the therapeutically active agent or agent solvent may or may not be a solvent for the polymer or matrix or a combination thereof.

One or more solvents may be used in accordance with the present invention. Suitable solvents for use in the method of the present invention include solvents miscible or highly soluble in a selected non-solvent such as for example but not limited to acetone, acetonitrile, ethanol, isopropyl alcohol, dimethyl sulfoxide, dimethyl formamide, tetrahydrofuran and dioxane. Preferred solvents include acetone and acetonitrile because their relatively strong solvent nature allows for particle formation of many materials. The volume of one or more solvents used in the present method is typically in the range of about 5 percent to about 50 percent.

One or more non-solvents may be used in accordance with the present invention. Suitable non-solvents for use in the method of the present invention include for example but are not limited to water, ethanol and methanol. The preferred non-solvent is water because of the ability to use secondary factors such as for example pH to further control particle formation processes. The volume of one or more non-solvents used in the present method is typically in the range of about 50 percent to about 75 percent of the solvent/non-solvent system.

Solvent/non-solvent systems of the present invention may include one or more solvents and/or one or more non-solvents. Suitable solvent/non-solvent systems for use in the method of the present invention include for example but are not limited to acetone/water and acetonitrile/water. The preferred solvent and non-solvent system is acetone/water because phase separation can be controlled through a wide range of ternary agent concentrations. The volume of solvent/non-solvent system used in the present method is typically in the range of about 10 mL to about 100 L.

One or more ternary agents may be used in accordance with the present invention. Suitable ternary agents for use in the method of the present invention include for example but are not limited to ammonium azide, ammonium bisulfite, barium acetate hydrate, barium hypophosphate, cadmium chloride, calcium acetate dihydrate, calcium chromate, calcium ethyl methyl acetate, cobalt perchlorate, iron perchlorate hexahydrate, lead chlorate hydrate, lithium hydroxide monohydrate, lithium sulfate, lithium sulfite monohydrate, potassium carbonate, potassium chloride, potassium phosphate, sodium selenate, sodium stannate (hydroxo), sodium phosphate, strontium acetate and yttrium chloride. Preferred ternary agents include sodium chloride and sodium bromide because of their strong interactions with non-solvents such as for example water, leading to solvent phase separation. The volume of one or more ternary agents used in the present method is typically in the range of about 0.1 M to about 10 M.

One or more polymers may be used in accordance with the present invention. Suitable polymers for use in the method of the present invention include for example but are not limited to polyesters, polyanhydrides, polyorthoesters, polyurethanes, polyethylene and its derivatives, all acrylate-based polymers including poly(acrylic acid), poly(methyl methacrylate) and poly(2-hydroxyethyl methacrylate), poly(N-vinylpyrrolidone) and polyethylenimine. Preferred polymers include polyurethanes and polysaccharides because the same allow optimal particle forming properties to be included in the material selection. The volume of one or more polymers used in the present method is typically in the range of about 0.01 percent w/v solvent/non-solvent system to about 1.0 percent w/v solvent/non-solvent system.

One or more matrices may be used in accordance with the present invention. Suitable matrices for use in the method of the present invention include for example but are not limited to trehalose, dextrose, triethanolamine, tetraethyl orthosilicate and calcium carbonate. Preferred matrices include trehalose, dextrose and triethanolamine because of their lyoprotectant and ionic interaction properties. The volume of one or more matrices used in the present method is typically in the range of about 0.01 percent w/v solvent/non-solvent system to about 1.0 percent w/v solvent/non-solvent system.

One or more solvents having temperature dependent solubility may be used in accordance with the present invention. Suitable solvents having temperature dependent solubility for use in the method of the present invention include for example but are not limited to acetone, acetonitrile, ethanol, isopropyl alcohol, dimethyl sulfoxide, dimethyl formamide, tetrahydrofuran and dioxane. Preferred solvents having temperature dependent solubility include acetone and acetonitrile because of their relatively strong solvating power. The volume of one or more solvents having temperature dependent solubility used in the present method is typically in the range of about 5.0 percent v/v of the solvent/non-solvent system to about 50 percent v/v of the solvent/non-solvent system.

One or more surfactants may be used in accordance with the present invention. Suitable surfactants for use in the method of the present invention include for example but are not limited to poly(N-vinylpyrrolidone), poly(ethylene oxide)/poly(propylene oxide) triblock copolymers, Tweens, Sorbitans and triacyl glycerols. Preferred surfactants include poly(ethylene oxide)/poly(propylene oxide) triblock copolymers because the broad range of polymers allows for the selection of an optimal stabilizing agent. The volume of one or more surfactants used in the present method is typically in the range of about 0.1 percent w/v of the solvent/non-solvent system to about 5.0 percent w/v of the solvent/non-solvent system.

One or more agent solvents may be used in accordance with the present invention. Suitable agent solvents for use in the method of the present invention include polar charged, polar uncharged, polar, charged or neutral solvents, such as for example but not limited to chloroform, carbon tetrachloride, 1,2-dichloroethane, dichloromethane, ethyl acetate and toluene. The preferred agent solvent is ethyl acetate because of its solubility in many non-solvents. The volume of one or more agent solvents used in the present method is typically in the range of about 0.01 percent of the solvent/non-solvent system to about 10.0 percent of the solvent/non-solvent system.

One or more therapeutic agents may be used in accordance with the present invention. Suitable therapeutic agents for use in the method of the present invention include for example but are not limited to beta-blockers, anti-glaucoma agents such as for example but not limited to the beta blockers timolol maleate, betaxolol and metipranolol, mitotics such as for example but not limited to pilocarpine, acetylcholine chloride, isofluorophate, demacarium bromide, echothiophateiodide, phospholine iodide, carbachol and physostigimine, epinephrine and salts such as for example but not limited to dipivefrin hydrochloride, dichlorphenamide, acetazolamide and methazolamide, anti-cataract and anti-diabetic retinopathy agents such as for example but not limited to the aldose reductase inhibitors tolrestat, lisinopril, enalapril and statil, thiol cross-linking agents, anticancer agents such as for example but not limited to retinoic acid, methotrexate, adriamycin, bleomycin, triamcinoline, mitomycin, cisplatinum, vincristine, vinblastine, actinomycin-D, ara-c, bisantrene, activated cytoxan, melphalan, mithramycin, procarbazine and tamoxifen, immune modulators, anti-clotting agents such as for example but not limited to tissue plasminogen activator, urokinase and streptokinase, anti-tissue damage agents such as for example but not limited to superoxide dismutase, proteins and nucleic acids such as for example but not limited to mono- and poly-clonal antibodies, enzymes, protein hormones and genes, gene fragments and plasmids, steroids, particularly anti-inflammatory or anti-fibrous agents such as for example but not limited to loteprednol, etabonate, cortisone, hydrocortisone, prednisolone, prednisome, dexamethasone, progesterone-like compounds, medrysone (HMS) and fluorometholone, non-steroidal anti-inflammatory agents such as for example but not limited to ketrolac tromethamine, dichlofenac sodium and suprofen, antibiotics such as for example but not limited to loridine (cephaloridine), chloramphenicol, clindamycin, amikacin, tobramycin, methicillin, lincomycin, oxycillin, penicillin, amphotericin B, polymyxin B, cephalosporin family, ampicillin, bacitracin, carbenicillin, cepholothin, colistin, erythromycin, streptomycin, neomycin, sulfacetamide, vancomycin, silver nitrate, sulfisoxazole diolamine and tetracycline, other antipathogens including anti-viral agents such as for example but not limited to idoxuridine, trifluorouridine, vidarabine (adenine arabinoside), acyclovir (acycloguanosine), pyrimethamine, trisulfapyrimidine-2, clindamycin, nystatin, flucytosine, natamycin, and miconazole, piperazine derivatives such as for example but not limited to diethylcarbamazine, and cycloplegic and mydriatic agents such as for example but not limited to atropine, cyclogel, scopolamine, homatropine and mydriacyl.

Other therapeutically active agents or drugs include anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

Therapeutically active agents or drugs of particular interest include hydrocortisone (5-20 mcg/l as plasma level), gentamycin (6-10 mcg/ml in serum), 5-fluorouracil (˜30 mg/kg body weight in serum), sorbinil, interleukin-2, phakan-a (a component of glutathione), thioloa-thiopronin, bendazac, acetylsalicylic acid, trifluorothymidine, interferon (α, β and γ), immune modulators such as for example but not limited to lymphokines and monokines and growth factors. Preferred therapeutic agents include proteins and nucleic acids because this method is relatively mild allowing high retention of biomolecule activity. The volume of one or more therapeutic agents used in the present method is typically in the range of about 1.0 percent to about 45 percent.

The present method is useful for the production of low-obscuration image transmitting ocular therapeutic particulate formulations through the use of ternary agent and temperature alteration induced immiscibility as is described in more detail below. A solution of one or more non-solvents, one or more ternary agents, and one or more surfactants are prepared at a starting temperature. One or more polymers, one or more matrices or combinations of one or more polymers and one or more matrices are dissolved in a selected solvent or solvent system. One or more desired therapeutically active agents are dissolved in a selected agent solvent or agent solvent system. Either the polymer and/or matrix solution is mixed with the therapeutically active agent solution before addition to the non-solvent solution, or the two are added separately to the non-solvent solution. The temperature of the solution of non-solvent(s), ternary agent(s), surfactant(s), polymer and/or matrix solution and therapeutically active agent solution is either increased or decreased to reduce the solubility of the solvents in the non-solvent solution. Changes in temperature may be performed rapidly or slowly, continuously or stepwise, or linearly or non-linearly. With the associated change in temperature, solvent(s) form emulsions with the non-solvent solution. Emulsified solvents may consist of elements of the solvent system for polymer or matrix or combinations thereof, and/or elements of the solvent system for the active therapeutic agent(s). Emulsified solvent being a better solvent for polymer or matrix or a combination thereof or for one or more active agents than the solvent and non-solvent system, therapeutically active or inactive agents preferentially partition into the better solvent.

Emulsification may be controlled to preferentially force one solvent out of the non-solvent solution to effect formation of a core of material or regions with different relative amounts materials or densities of a single material, therapeutically active or inactive. Temperature alteration profile may be controlled to produce a core of material or regions with different relative amounts materials or densities of a single material, therapeutically active or inactive. Because all emulsified droplets form from the same solution and grow under similar conditions, a narrow particulate size distribution can be achieved for particulates from about 1 nm to about 5 mm in size.

Following or concurrent with temperature alteration is solvent removal by alteration of pressure or vapor phase composition. Solvent removal may accompany different stages of nanoparticle or microparticle formation. Removal may be controlled to remove selected solvent or solvents or part of selected solvent or solvents. The timing of temperature change and solvent removal is controlled to produce particulates in the size range from 1 nm to 5 mm. In the final phase, solvent removal is extensive enough to produce hardened polymeric or matrix particulates.

The method of the present invention is described in still greater detail in the following example.

### EXAMPLE 1

### Poly(lactic-co-glycolic acid) Nanospheres and Microspheres Prepared Using A Water, Acetonitrile, and Sodium Chloride System

In a specific embodiment of the method of the present invention, the non-solvent is water, the ternary agent is sodium chloride, the surfactant is a poly(ethylene oxide)-poly(propylene oxide)-poly(ethylene oxide) triblock copolymer commercially available under the trade name Pluronic F127™ (BASF Wyandotte Corp., Wyandotte, Mich.). Acetonitrile is used as the polymer and therapeutic agent solvent. The polymer is a 50/50 copolymer of lactic and glycolic acids (PLGA) with a molecular weight of approximately 12,300. Evaluating the effect of temperature change on acetonitrile solubility in the non-solvent solution, it was determined that acetonitrile possesses a solubility maximum in a solution of sodium chloride in water at 45° C. Furthermore, it was determined that the change in solubility over the temperature range of −10° C. to 55° C., the change in the solubility was inversely proportional to the salt concentration from 5 M to 1 M. The greatest change in the solubility was for a 40 percent solution of acetonitrile in 1 M sodium chloride solution in water. At 35° C., 40 percent acetonitrile was completely soluble in the non-solvent salt solutions, but the volume occupied by the acetonitrile phase was approximately 18.6 percent of the total volume when the temperature was reduced to −10° C. After mixing the non-solvent solution, 1 M sodium chloride and 1 percent Pluronic F127™ in water, a 0.25 percent solution of PLGA was made in acetonitrile. A volume of the polymer-containing solvent solution was added to the non-solvent solution in a ratio of 4:6 by volume. The combination of the two solutions was heated in a water bath to 35° C. After reaching the temperature required to fully dissolve the solvent in the non-solvent solution, the entire volume was transferred to a vacuum flask at 10° C. and stirred. When it was confirmed that the temperature of the solvent and non-solvent solution was 10° C., vacuum was drawn to remove the acetonitrile. The experiment was repeated. However, the temperature was only decreased to 20° C. The resulting hardened PLGA particles were collected by filtration through a 100 nm filter, rinsed and dried. Analysis using dynamic light scattering, scanning electron microscopy, and atomic force microscopy confirmed that different sized particles were created.

The greater the volume of solvent emulsified in the non-solvent, the smaller the particle size. Assuming emulsion droplets originate from a continuous solution, all droplets must begin forming at the same size. Rapidly reducing the temperature induces many more emulsification events as more solvent agglomerates out of solution. For a constant amount of polymer soluble in the solvent, the more emulsion droplets that form, the smaller the mass of polymer in each droplet. After droplets are hardened, the smaller mass dissolved in the droplet results in a smaller final particle. Varying the amount of polymer dissolved in the solvent and the amount of the temperature change varies the amount of polymer that dissolves in the emulsion droplet and the number of emulsion droplets formed, respectively. Additionally, starting with a different salt concentration would alter the amount of solvent that emulsifies on temperature alteration and ultimately particle size. The elements of this process provide excellent control of the formation of particles in the size range from about 1 nm to about 1 μm.

Ocular therapeutic particulate formulations produced in accordance with the present method may be used as customary in the field of ophthalmology. Such uses include for example but not limited to ocular topical applications such as for example but not limited to drops, gels or suspensions for external delivery to the eye, and parenteral applications such as for example but not limited to hypodermic injection into the tissues of the eye for example but not limited to vitreous humor, aqueous humor, cornea, sclera, retina and choroids.

While there is shown and described herein low-obscuration image transmitting ophthalmic therapeutic particulate formulations and methods of making and using the same, it will be manifest to those skilled in the art that various modifications may be made without departing from the spirit and scope of the underlying inventive concept. The present invention is likewise not intended to be limited to particular treatments described herein except insofar as indicated by the scope of the appended claims.

We claim:

1. A low-obscuration image transmitting ocular composition comprising:
particulates of about 0.10 to about 500 nm diameter at a concentration of about 0.01 to about 500 mg/ml for ocular therapeutic use.

2. A low-obscuration image transmitting ocular composition comprising:
particulates of about 0.10 to about 500 nm diameter having a real refractive index of about 1.0 to about 1.8 at a concentration of about 0.01 to about 500 mg/ml for ocular use.

3. A low-obscuration image transmitting ocular composition comprising:
particulates of about 0.10 to about 500 nm diameter having an imaginary refractive index of less than about 0.20 or greater than about 0.30 at a concentration of about 0.01 to about 500 mg/ml for ocular use.

4. A low-obscuration image transmitting ocular composition comprising:
particulates of less than about 0.20 or greater than about 0.30 imaginary refractive index at a concentration of about 0.01 to about 500 mg/ml for ocular use.

5. A low-obscuration image transmitting ocular composition comprising:
particulates of about 1.0 to about 1.8 real refractive index at a concentration of about 0.01 to about 500 mg/ml for ocular use.

6. The composition of claim 1, 2, 3, 4 or 5 further comprising a secondary material to alter said particulates for ocular use.

7. The composition of claim 1, 2 or 5 further comprising a secondary material to alter imaginary refractive index of said particulates for ocular use.

8. The composition of claim 3 or 4 further comprising a secondary material to achieve said imaginary refractive index of said particulates.

9. The composition of claim 1, 3 or 4 further comprising a secondary material to alter real refractive index of said particulates for ocular therapeutic use.

10. The composition of claim 2 or 5 further comprising a secondary material to achieve said real refractive index of said particulates.

11. The composition of claim 1, 2 or 3 further comprising a secondary material to achieve said diameter of said particulates for ocular therapeutic use.

12. The composition of claim 4 or 5 further comprising a secondary material to alter diameters of said particulates for ocular therapeutic use.

13. The composition of claim 6, 7, 8, 9, 10, 11 or 12 wherein said one or more secondary materials are selected from the group consisting of polyolefins, polyethers, polyesters, polyanhydrides, polyamides, polyorthoesters, polyacrylates, polysulfones, polysiloxanes, polysaccharides, polyvinylpyrrolidone, polyols, alcohols, water, and inorganic compounds.

14. An ocular composition comprising:
particulates of 10 mg/ml concentration having, while in an eye or surrounding tissues, less than 1 dB variation due to light scattering over 0.050 in the eye.

15. The composition of claim 1, 2, 3, 4, 5, 6, 7, 8, 9, 10, 11, 12, 13 or 14 further comprising a therapeutically active agent.

16. The composition of claim 1, 2, 3, 4, 5, 6, 7, 8, 9, 10, 11, 12, 13 or 14 wherein said particulates include a therapeutically active agent.

17. A method of using the composition of claim 1, 2, 3, 4, 5, 6, 7, 8, 9, 10, 11, 12, 13, 14, 15 or 16 comprising:
administering topically said composition to tissue.

18. A method of using the composition of claim 1, 2, 3, 4, 5, 6, 7, 8, 9, 10, 11, 12, 13, 14, 15 or 16 comprising:
administering parenterally said composition to tissue.
